(19) United States
(12) Patent Application Publication
(10) Pub. No.: US 2010/0105089 A1
(43) Pub. Date: Apr. 29, 2010

(54) QUANTUM DOT BIOTAGS

(75) Inventor: LETHA J. SOOTER, MORGANTOWN, WV (US)

Correspondence Address: U S ARMY RESEARCH LABORATORY;ATTN: RDRL-LOC-I, 2800 POWDER MILL RD, ADELPHI, MD, 20783-1197, US

(73) Assignee: UNITED STATES OF AMERICA AS REPRESENTED BY THE SECRETARY OF THE ARMY

(21) Appl. No.: 12/571,004

(22) Filed: Sep. 30, 2009

Related U.S. Application Data
(60) Provisional application No. 61/136,768, filed on Oct. 1, 2008.

(51) Int. Cl. G01N 33/53; C08L 101/00; C08L 89/00; C07K 1/107; C07H 1/00; C12N 1/00; C12Q 1/02

(52) U.S. Cl. 435/7.94; 525/50; 525/54.2; 525/54.1; 530/345; 530/402; 536/25.32; 435/243; 435/29; 977/774

(57) ABSTRACT

A biotag that includes a biomolecule that includes a bio polymer sequence; an attaching polymer attached at an endpoint of the biopolymer sequence; and a quantum dot bound to the attaching polymer causing the attaching polymer to become fluorescently labeled is provided. Suggested biomolecules include, but are not limited to, peptides, proteins, amino acids, nucleic acids, deoxyribonucleic acids, ribonucleic acids, and peptide nucleic acids. The biopolymer sequence may be or otherwise include a protein sequence. The biomolecule may be located inside a cell, outside a cell, or on a cell. Desirably, a quantum dot is bound to an attaching polymer and, thus the biomolecule, without using any of thiol chemistries, 1-Ethyl-3-[3-dimethylaminopropyl]carbodiimide/N-hydroxysuccinimide hydrochloride (EDC/NHS) chemistries, and biotin/avidin chemistries. In certain desirable embodiments, the attaching polymer is attached at an endpoint of a biopolymer sequence using any of synthesizing, replicating, transcribing, translating, and expressing the attaching polymer at any of a C-terminal end or an N-terminal end of the biopolymer sequence.

### CROSS REFERENCE TO RELATED APPLICATION

This application claims the benefit of U.S. Provisional Patent Application No. 61/136,768 filed on Oct. 1, 2008. The complete disclosure of which is hereby incorporated by reference herein in its entirety.

### GOVERNMENT INTEREST

The embodiments herein may be manufactured, used, and/or licensed by or for the United States Government without the payment of royalties thereon.

### TECHNICAL FIELD

The embodiments herein generally relate to biomolecule labeling, and more particularly to quantum dots used in biomolecule labeling.

### BACKGROUND

A semiconductor having excitons confined in all three spatial dimensions is called a quantum dot. Quantum dots have material properties that are between those of bulk semiconductors and those of discrete molecules. Moreover, quantum dots are robust fluorophores and can be defined as high quality nanocrystals made from a variety of semiconductor materials, for example, CdS, ZnS, ZnSe, and other semiconductor materials. Quantum dots absorb and emit light of different wavelengths according to their size and composition, have a broad range of excitation wavelengths, and do not photobleach. As quantum dot labeling products are becoming more available, they are being used increasingly in the laboratory. For example, quantum dots can be used for drug delivery and in a variety of medical imaging techniques. For example, monoclonal antibodies against an Epidermal Growth Factor Receptor (EGFR) can be attached to quantum dots. Cells which overproduce EGFR are more likely to become malignant. They use the fluorescing quantum dots to identify these potentially cancerous cells. Current methods of labeling biomolecules rely upon conjugation chemistry to link a biomolecule with a functionalized outer coating on the quantum dot.

### SUMMARY

In view of the foregoing, an embodiment herein provides a method of forming a composition of matter, the method comprising providing a biomolecule comprising a biopolymer sequence; attaching an attaching polymer at a point of the biopolymer sequence; and binding a quantum dot to the attaching polymer causing the polymer and, thus the modified biomolecule, to become fluorescently labeled. The starting biomolecule may be or include any of a peptide, a protein, an amino acid, a nucleic acid, a deoxyribonucleic acid (DNA), a ribonucleic acid (RNA), and a peptide nucleic acid (PNA). The biopolymer sequence may be or otherwise include a protein sequence. The binding comprises incubating the attaching polymer with the quantum dot. The modified biomolecule may be located inside of a cell, outside a cell, or on a cell. Preferably, the binding occurs without any of thiol chemistries, 1-Ethyl-3-[3-dimethylaminopropyl]carbodiimide/N-hydroxysuccinimide hydrochloride (EDC/NHS) chemistries, and biotin/avidin chemistries. The attaching may comprise any of synthesizing, replicating, transcribing, translating, and expressing the attaching polymer at any of a C-terminal end or an N-terminal end of the biopolymer sequence. Furthermore, the composition of matter is preferably used as a biotag. The method may further include using the biotag in any of a sandwich style assay, direct fluorescent labeling assay, fluorescence activated cell sorting, cell identification, and in vivo protein localization.

Another embodiment provides a biotag that includes a biomolecule that includes a biopolymer sequence; an attaching polymer attached at some point along the biopolymer sequence; and a quantum dot bound to the attaching polymer causing the attaching polymer and, thus, the modified biomolecule to become fluorescently labeled. The biomolecule may be or otherwise include any of a peptide, a protein, an amino acid, a nucleic acid, a deoxyribonucleic acid, a ribonucleic acid, and a peptide nucleic acid. The biopolymer sequence may be or otherwise include a protein sequence. The biomolecule may be located inside, outside, or on a cell. Preferably, the quantum dot is bound to the attaching polymer without using any of thiol chemistries, 1-Ethyl-3-[3-dimethylaminopropyl]carbodiimide/N-hydroxysuccinimide hydrochloride (EDC/NHS) chemistries, and biotin/avidin chemistries. The attaching polymer is attached at a point on the biopolymer sequence, preferably at an endpoint of the biopolymer sequence, using any of synthesizing, replicating, transcribing, translating, and expressing the attaching polymer at any of a C-terminal end or an N-terminal end of the biopolymer sequence.

Another embodiment provides a composition of matter that includes a biomolecule that includes a biopolymer sequence, wherein the biomolecule is or otherwise includes any of a peptide, a protein, an amino acid, a nucleic acid, a deoxyribonucleic acid, a ribonucleic acid, and a peptide nucleic acid; an attaching polymer attached at any point from a beginning to an end of the biopolymer sequence using any of synthesizing, replicating, transcribing, translating, and expressing the attaching polymer at an endpoint of the biopolymer sequence, wherein the endpoint comprises any of a C-terminal end or an N-terminal end of the biopolymer sequence; and a quantum dot bound to the attaching polymer causing the attaching polymer to become fluorescently labeled, wherein the quantum dot is bound to the attaching polymer without using any of thiol chemistries, 1-Ethyl-3-[3-dimethylaminopropyl]carbodiimide/N-hydroxysuccinimide hydrochloride (EDC/NHS) chemistries, and biotin/avidin chemistries.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a schematic diagram of selecting biotags according to an embodiment herein;

FIG. 2 illustrates a schematic diagram of an apparatus for selecting biotags according to an embodiment herein;

FIG. 3 illustrates a schematic diagram of using biotags according to an embodiment herein; and

FIG. 4 illustrates a flow diagram according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

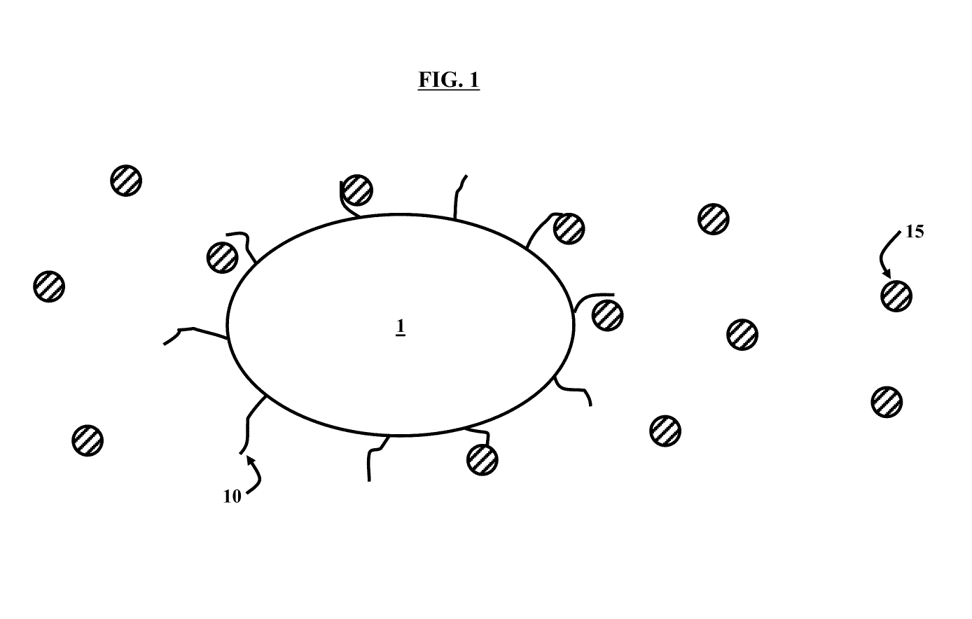

The embodiments herein provide quantum dots that are bound to biomolecules, for example a protein, to provide a fluorescently tagged biomolecule. Referring now to the drawings, and more particularly to FIGS. 1 through 4, where similar reference characters denote corresponding features consistently throughout the figures, there are shown preferred embodiments.

In the embodiment illustrated herein a biomolecule 5 comprising a biopolymer sequence (for example, an antibody) is modified by attaching at least one attaching polymer 10 at some point along the biopolymer sequence, preferably an endpoint of the biopolymer sequence. As used herein, the term “attaching polymer” includes any synthetic or natural polymer including, but not limited to biopolymers, that can be used to attach a quantum dot to a biopolymer sequence in a biomolecule, for example attaching a quantum dot to any of a C-terminal or an N-terminal end of a biopolymer sequence via an attaching polymer.

At least one quantum dot 15 is bound to the provide polymer 10 causing the provide polymer 10 and modified biomolecule 5 to become fluorescently labeled. Quantum dots 15 emit light in various visible and infrared wavelengths including, but not limited to, wavelengths corresponding to the colors, red, red/orange, orange, yellow, fluorescent yellow, green, and blue. By expressing a quantum dot 15 at a point along the attaching polymer sequence, preferably, a terminus of the attaching polymer sequence, biochemists do not have to purchase modified quantum dots or perform any difficult conjugation chemistries. Quantum dots 15 can also been functionalized with biotin or streptavidin on their outer surface. In vitro selections can be used to isolate biomolecules 5, for example nucleic acid, amino acid, peptides, protein, DNA, RNA, and PNA that bind quantum dots 15.

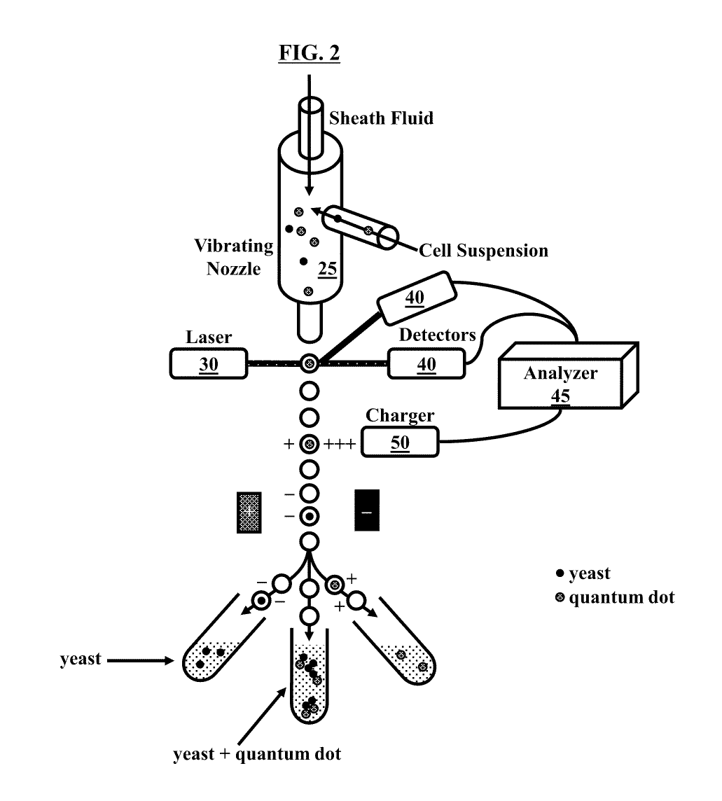

Referring to FIG. 1, a yeast cell 1 that includes peptide 10 links is incubated with quantum dots 15 of choice. Desirably, quantum dots 15 are selected to bind to peptides 10 of a particular sequence. FIG. 2 illustrates an apparatus 100 for selecting biotags. Fluorescence activated cell sorting isolates the yeast 1 displaying peptides 10 that bind the quantum dots 15. For example, isolation of DNA from the yeast cell 1 reveals the peptide sequence that binds the quantum dots 15. Accordingly, the peptide 10 with this sequence is the biotag. As shown in the apparatus of FIG. 2, sheath fluid that includes both yeast cells 1 and quantum dots 15 is introduced in a vibrating nozzle 25 where cell suspension occurs. A laser 30 is applied to the extracted fluid where detectors 40 detect the presence of the quantum dots 15, which are then analyzed by analyzer 45. Next a charger 50 applies a charge to the fluid. The yeast cells 1 and quantum dots 15 may be selectively isolated according to the charge (+ or −) and a mixed solution of yeast cells 1 and quantum dots 15 is generated.

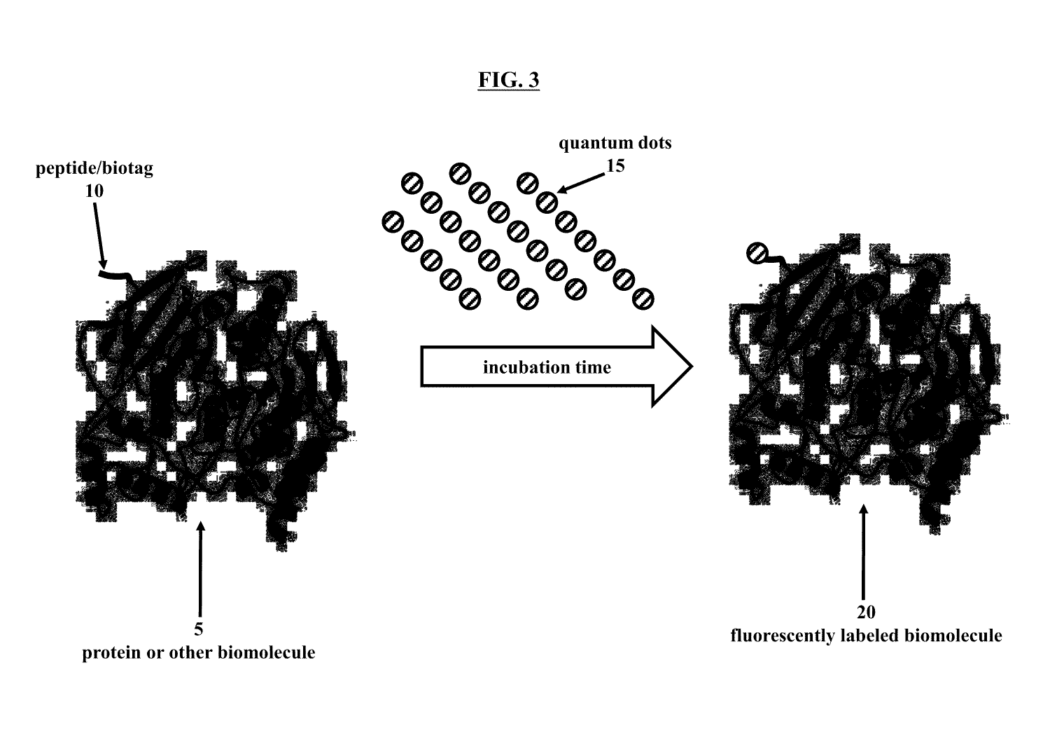

As shown in FIG. 3, a peptide 10 is attached at the beginning or end of a biopolymer sequence of a biomolecule 5. Next, the tagged protein is incubated with the proper quantum dot(s). The biomolecule 5 becomes fluorescently labeled 20 by binding a quantum dot 15 to the peptide 10. This modified biomolecule is useful for biochemistry applications and for such things as the visualization of protein localization within cells.

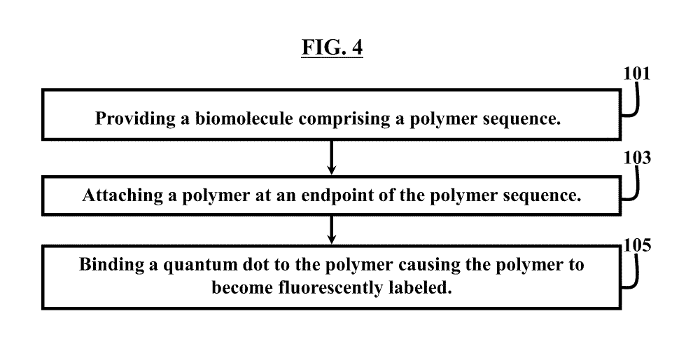

Referring now to FIG. 4, with reference to FIGS. 1 through 3, is a flowchart illustrating a method of forming a composition of matter, the method including providing (101) a biomolecule 5 that includes at least one polymer sequence; attaching (103) an attaching polymer 15 at some point along of the biopolymer sequence, for example an endpoint of the biopolymer sequence; and binding (105) a quantum dot 15 to the attaching polymer 15 causing the attaching polymer 15 and, thus, the modified biomolecule to become fluorescently labeled 20. Suggested biomolecules include, but are not limited to, peptides, proteins, nucleic acids, deoxyribonucleic acids, ribonucleic acids, and peptide nucleic acids. The biopolymer sequence may be or otherwise include a protein sequence. The binding process (105) comprises incubating the attaching polymer 10 with one or more quantum dots 15. The biomolecule 5 may be located inside of a cell (for example, a yeast cell), outside a cell, or on a cell at the time of labeling. Preferably, the binding occurs without any of thiol chemistries, 1-Ethyl-3-[3-dimethylaminopropyl]carbodiimide/N-hydroxysuccinimide hydrochloride (EDC/NHS) chemistries, and biotin/avidin chemistries. The attaching (103) of the attaching polymer to the biomolecule may include any of synthesizing, replicating, transcribing, translating, and expressing the attaching polymer at any of a C-terminal or an N-terminal end of the biopolymer sequence. In other words, the peptides 10 can be expressed as C-terminal or N-terminal tags on proteins of interest 5 in order to attach them to a quantum dot 15.

Furthermore, the composition of matter preferably includes or is used as a biotag. The method may further include using the biotag in any of a sandwich style assay, direct fluorescent labeling assay, fluorescence activated cell sorting, cell identification, and in vivo protein localization. The biomolecules 5 can bind specific quantum dots 15 with high affinity and specificity, and can differentiate between quantum dots 15 based upon size, material, coatings, and crystal structure. The biomolecules 5 can be expressed or synthesized as biotags on other molecules of interest in order to attach them to a quantum dot 15. The embodiments herein allow unmodified quantum dots to be used, which are less expensive and easier to produce. The embodiments herein save time and money through fewer chemical modification and conjugation chemistries.

Other suggested components and methods that may be used in conjunction with the present invention are described in “DNA Aptamer-Passivated Nanocrystal Synthesis: A Facile Approach for Nanoparticle-Based Cancer Cell Growth Inhibition”, Small Volume 5, Issue 6, Date: Mar. 20, 2009, Pages: 672-675 by Jong Hyun Choi, Kok Hao Chen, Jae-Hee Han, Amanda M. Chaffee and Michael S. Strano and “High Affinity scFv-Hapten Pair as a Tool for Quantum Dot Labeling and Tracking of Single Proteins in Live Cells” Nano Lett., 2008, 8 (12), pp 4618-4623 Publication Date (Web): Nov. 20, 2008 by Gopal Iyer, Xavier Michalet, Yun-Pei Chang, Fabien F. Pinaud, Stephanie E. Matyas, Gregory Payne and Shimon Weiss both of which are hereby incorporated by reference herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method of forming a composition of matter, said method comprising:
providing a biomolecule comprising a biopolymer sequence;
attaching a attaching polymer at some point along said polymer sequence; and
binding a quantum dot to said attaching polymer causing said attaching polymer to become fluorescently labeled.

2. The method of claim 1, wherein said biomolecule comprises any of a peptide, a protein, an amino acid, a nucleic acid, a deoxyribonucleic acid, a ribonucleic acid, and a peptide nucleic acid.

3. The method of claim 1, wherein said biopolymer sequence comprises a protein sequence.

4. The method of claim 1, wherein said binding comprises incubating said attaching polymer with said quantum dot.

5. The method of claim 1, wherein said biomolecule is located inside of a cell.

6. The method of claim 1, wherein said biomolecule is located outside of a cell.

7. The method of claim 1, wherein said biomolecule is located on a cell.

8. The method of claim 1, wherein said binding occurs without any of thiol chemistries, 1-Ethyl-3-[3-dimethylaminopropyl]carbodiimide/N-hydroxysuccinimide hydrochloride (EDC/NHS) chemistries, and biotin/avidin chemistries.

9. The method of claim 1, wherein said attaching comprises any of synthesizing, replicating, transcribing, translating, and expressing said attaching polymer at any of a C-terminal end or an N-terminal end of said biopolymer sequence.

10. The method of claim 1, wherein said composition of matter is used as a biotag.

11. The method of claim 9, further comprising using said biotag in any of a sandwich style assay, direct fluorescent labeling assay, fluorescence activated cell sorting, cell identification, and in vivo protein localization.

12. A biotag comprising:
a biomolecule comprising a biopolymer sequence;
an attaching polymer attached at an endpoint of said biopolymer sequence; and
a quantum dot bound to said attaching polymer causing said attaching polymer to become fluorescently labeled.

13. The biotag of claim 12, wherein said biomolecule comprises any of a peptide, a protein, an amino acid, an nucleic acid, a deoxyribonucleic acid, a ribonucleic acid, and a peptide nucleic acid.

14. The biotag of claim 12, wherein said bio polymer sequence comprises a protein sequence.

15. The biotag of claim 12, wherein said biomolecule is located inside of a cell.

16. The biotag of claim 12, wherein said biomolecule is located outside of a cell.

17. The biotag of claim 12, wherein said biomolecule is located on a cell.

18. The biotag of claim 12, wherein said quantum dot is bound to said polymer without using any of thiol chemistries, 1-Ethyl-3-[3-dimethylaminopropyl]carbodiimide/N-hydroxysuccinimide hydrochloride (EDC/NHS) chemistries, and biotin/avidin chemistries.

19. The biotag of claim 12, wherein said attaching polymer is attached at said endpoint of said biopolymer sequence using any of synthesizing, replicating, transcribing, translating, and expressing said attaching polymer at any of a C-terminal end or an N-terminal end of said biopolymer sequence.

20. A composition of matter comprising:
a biomolecule comprising a biopolymer sequence, wherein said biomolecule comprises any of a peptide, a protein, an amino acid, a nucleic acid, a deoxyribonucleic acid, a ribonucleic acid, and a peptide nucleic acid;
an attaching polymer attached at any of a beginning and an end of said biopolymer sequence using any of synthesizing, replicating, transcribing, translating, and expressing said attaching polymer at an endpoint of said biopolymer sequence, wherein said endpoint comprises any of a C-terminal end or an N-terminal end of said biopolymer sequence; and
a quantum dot bound to said attaching polymer causing said polymer to become fluorescently labeled, wherein said quantum dot is bound to said attaching polymer without using any of thiol chemistries, 1-Ethyl-3-[3-dimethylaminopropyl]carbodiimide/N-hydroxysuccinimide hydrochloride (EDC/NHS) chemistries, and biotin/avidin chemistries.
